(19) United States
(12) Patent Application Publication
(10) Pub. No.: US 2023/0263734 A1
(43) Pub. Date: Aug. 24, 2023

(54) SOLID DOSAGE FORMS OF PALBOCICLIB

(71) Applicant: AIZANT DRUG RESEARCH SOLUTIONS PRIVATE LIMITED, Hyderabad (IN)

(72) Inventors: Bithunshal USHA BALAKRISHNAN, Quthbullapur Mandal, Hyderabad (IN); Ramdas MANAKKOTE, Quthbullapur Mandal, Hyderabad (IN); Srinivasa Rajasekara RUDRARAJU VARMA, Quthbullapur Mandal, Hyderabad (IN)

(21) Appl. No.: 18/019,582

(22) PCT Filed: Aug. 3, 2021
(86) PCT No.: PCT/IN2021/050746

§ 371 Date: Feb. 3, 2023

(30) Foreign Application Priority Data
Aug. 5, 2020 (IN) 202041033503

(51) Int. Cl. A61K 9/20; A61K 31/519; A61K 31/198; A61K 38/06

(52) U.S. Cl. CPC A61K 9/2054; A61K 31/519; A61K 31/198; A61K 9/2027; A61K 9/2013; A61K 9/2009; A61K 38/063; A61K 9/0053

(57) ABSTRACT

The present invention relates to novel pharmaceutical compositions comprising palbociclib, as the as a pharmaceutically active compound. Specifically, the present invention relates to solid dosage forms of palbociclib comprising at least one compound selected from the group consisting of sulphur-containing amino acid or peptide, and at least one vitamin having antioxidant properties. The dosage forms described herein are chemically stable oral preparations having desirable pharmacokinetic characteristics and dissolution properties. The invention is also directed to the use of said pharmaceutical compositions as medicament, in particular for the treatment of cancer.

### CROSS REFERENCE

This application claims priority to an Indian provisional patent application no. 202041033503, filed Aug. 5, 2020, the contents of which are incorporated herein by reference in their entireties.

### FIELD OF THE INVENTION

The present invention relates to novel pharmaceutical compositions comprising palbociclib, as the as a pharmaceutically active compound. Specifically, the present invention relates to solid dosage forms of palbociclib comprising at least one compound selected from the group consisting of sulphur-containing amino acid or peptide, and at least one vitamin having antioxidant properties. The dosage forms described herein are chemically stable oral preparations having desirable pharmacokinetic characteristics and dissolution properties. The invention is also directed to the use of said pharmaceutical compositions as medicament, in particular for the treatment of cancer.

### BACKGROUND OF THE INVENTION

Palbociclib is a potent and selective inhibitor of CDK4 and CDK6, having the structure:

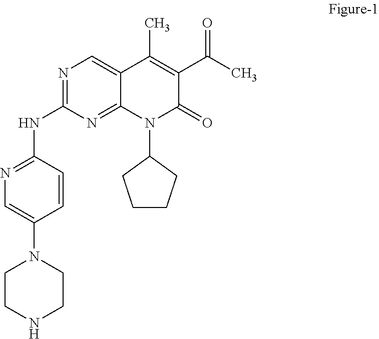 [SMILES: CC(=O)c1c(C)c2cnc(Nc3ccc(N4CCNCC4)cn3)nc2n(C2CCCC2)c1=O]

is named 6-acetyl-8-cyclopentyl-5-methyl-2-(5-piperazin-1-yl-pyridin-2-ylamino)-8H-pyrido[2,3-d]pyrimidin-7-one.

Palbociclib is approved in the United States for the treatment of hormone receptor (HR)-positive, human epidermal growth factor 2 (HER2)-negative advanced or metastatic breast cancer in combination with letrozole as initial endocrine therapy or in combination with fulvestrant following disease progression on endocrine therapy. The drug is sold by Pfizer under the trade name IBRANCE® in the form of immediate release (IR) capsule and tablet dosage forms comprising palbociclib as a free base for oral administration.

Palbociclib and pharmaceutically acceptable salts thereof are disclosed in WO 2003/062236 A1 which describes the preparation of palbociclib as its hydrochloride salt. WO 2005/005426 A1 describes the preparation of palbociclib free base and various mono- and di-acid addition salts thereof including various polymorphic forms of the isethionate salt. WO 2014/128588 A1 teaches that palbociclib free base provided by salt break procedures, e.g., as described in example 4 of WO 2005/005426 A1, was highly static prone and formed small primary particles, which agglomerated into large, hard agglomerates that were difficult to disperse by sieving and were unsuitable for further development (see page 2, lines 4 to 7). Therefore, WO 2014/128588 A1 provides palbociclib having larger primary particles and reduced specific surface area, which, according to the applicant, demonstrates improved physicochemical and manufacturability properties.

Due to its low solubility and high permeability palbociclib can be put into class II of the Biopharmaceutical Classification System (BCS). When administering such low soluble substances orally, the dissolution rate is the limiting factor during drug absorption. Hence, with regards to pharmaceutical processing, there is still a need for improving its dissolution rate which has a big impact on the bioavailability.

Moreover, the absorption and bioavailability of a therapeutic agent can be affected by numerous factors when dosed orally, including whether the subject is in a fed or fasted state, and the use of certain medications, such as proton pump inhibitors (PPIs) or H2 receptor antagonists, as well as certain medical conditions. Palbociclib presents a pH-dependent solubility, which decreases as the pH increases. In people having a pH higher than normal in the stomach, the dissolution of Palbociclib is thus impaired and this leads to a decreased bioavailability of the drug.

There remains a need to discover improved dosage forms of palbociclib having favorable dissolution and pharmacokinetic profiles, which also demonstrate good storage stability.

### SUMMARY OF THE INVENTION

Thus, the object of the present invention therefore is to provide a solid dosage form of palbociclib comprising at least one compound selected from the group consisting of sulphur-containing amino acid or peptide, and at least one vitamin having antioxidant properties.

Another object of the present invention is to provide stable, solid oral pharmaceutical composition comprising palbociclib, wherein the composition has comparable in-vitro dissolution profile similar to that of IBRANCE® tablets.

Another object of the present invention is to provide a stable pharmaceutical composition of palbociclib, where the composition has high drug loading, preferably more than 40% by weight of the composition.

Yet another object of the invention is to provide palbociclib formulations for oral administration, by a manufacturing process which is consistent and therefore feasible for industrial production, while maintaining stability and pharmaceutical equivalence to the reference listed drug.

### DETAILED DESCRIPTION OF THE INVENTION

In one aspect, present invention provides solid dosage forms of palbociclib comprising at least one compound selected from the group consisting of sulphur-containing amino acid or peptide, and at least one vitamin having antioxidant properties.

The inventors have surprisingly found that the solid dosage forms according to the present invention demonstrate excellent storage stability and provide substantially pH-independent delivery of palbociclib with no significant food effects or adverse interactions with PPIs.

Unless otherwise indicated herein, palbociclib refers the free base form of 6-acetyl-8-cyclopentyl-5-methyl-2-(5-piperazin-1-yl-pyridin-2-ylamino)-8H-pyrido[2,3-d]pyrimidin-7-one, which may be present in crystalline or amorphous form, or a mixture of amorphous and crystalline forms.

The sulphur-containing amino acid or peptide must act as a biologically active donor of the —S— or —SH group. Sulphur-containing amino acids are known to maintain or activate enzyme activity, thereby causing biochemical reactions involving SH groups. In the context of the present invention, sulphur-containing amino acids or peptides can be used as such or in the form of pharmaceutically acceptable salts or derivatives thereof.

Examples of these sulphur-containing amino acids or peptides include cysteine, methionine, aminoethylsulfonic acid (taurine), glutathione, cystine, homocysteine, homocystin, cysteine sulfinic acid, lanthionine, mixtures thereof, and pharmaceutically acceptable thereof. Including salts or derivatives. It is also possible to use mixed disulfides of any of the aforementioned compounds having thiol groups. However, among these disulfides, homogeneous disulfides are preferred. The use of one or more of these acids is preferred, particularly cysteine, methionine, taurine and glutathione, as well as their pharmaceutically acceptable salts or derivatives.

The amount of sulphur-containing amino acid will vary depending on the type, the combination selected and the route of application.

L-cysteine is one of the preferred sulphur-containing amino acids for use in the context of the present invention. For oral application, the daily dose for adults is usually in the range of 5-500 mg, preferably in the range of 10-250 mg.

Aminoethyl sulfonic acid is also known as taurine or 2-aminoethyl sulfonic acid, when applied orally, the daily dose for adults is 5-1000 mg, preferably 25-500 mg.

Glutathione is also γ-L-glutamyl-L-cysteinyl-glycine, when applied orally, the daily dose for adults is 5-1000 mg, preferably 25-600 mg.

In yet another embodiment the sulphur-containing amino acid in the composition can be a single sulphur-containing amino acid or a combination of sulphur-containing amino acid, combined in such a concentration to impart maximum therapeutic advantage.

The present pharmaceutical composition contains at least one antioxidant vitamin in addition to sulphur-containing amino acids or peptides. There are no specific restrictions on the types of antioxidant vitamins that can be combined with palbociclib, provided that the corresponding vitamins have antioxidant properties. In the context of the present invention, preferred examples include vitamin C, vitamin E, vitamin A, and such vitamin-like active substances.

In view of vitamin C/vitamin C-like active substances, at least one vitamin having antioxidant properties is preferably selected from one or more of the group consisting of: ascorbic acid, metal ascorbate, for example sodium ascorbate, potassium ascorbate, calcium ascorbate, magnesium ascorbate, aluminium ascorbate, ascorbic acid derivatives such as ascorbyl phosphate, especially sodium or potassium ascorbyl phosphate, magnesium ascorbyl phosphate, calcium ascorbyl phosphate, and aluminium ascorbyl phosphate, ascorbic sulfate such as disodium ascorbyl sulfate, potassium ascorbyl sulfate, magnesium ascorbyl sulfate, calcium ascorbyl sulfate, and ascorbyl aluminium sulfate, ascorbyl glucoside, for example, ascorbyl-2-glucoside, fatty acid ascorbyl glucoside, fatty acid ascorbyl, erythorbic acid (isoascorbic acid), and erythorbic acid metal salts such as sodium erythorbate.

Examples of vitamin E/vitamin E-like active substances are d-α-tocopherol, dl-α-tocopherol, d-α-tocopherol acetate, dl-α-tocopherol acetate, d-α-tocopherol succinate, dl succinate-α-tocopherol, dl-α-tocopherol calcium succinate, tocopherol nicotinate, vitamin E linoleate (preferably a mixture of tocopheryl esters, mainly tocopheryl linoleate), dl-β-tocopherol, dl-γ-tocopherol. D-δ-tocopherol, and natural mixed tocopherols.

Examples of vitamin A/vitamin A-like active substances are vitamin A, retinal acetate, retinol palmitate, retinol etretinate, vitamin A oil, cod liver oil, strong cod liver oil, and for example α-carotene, β-Carotene, γ-carotene, and lycopene can also be added.

One or more compounds of these antioxidant vitamins can be used to formulate an antioxidant vitamin containing the composition of the present invention.

In yet another embodiment the antioxidant vitamin in the composition can be a single antioxidant vitamin or a combination of antioxidant vitamin, combined in such a concentration to impart maximum therapeutic advantage.

In a second aspect, the present invention provides solid dosage forms of palbociclib comprising at least one compound selected from the group consisting of sulphur-containing amino acid or peptide, and at least one vitamin having antioxidant properties; wherein the sulphur-containing amino acid or peptide, and the vitamin having antioxidant properties are present in a specific ratio with respect to each other.

The pharmaceutical compositions of the present invention include the sulphur-containing amino acid or peptide and the antioxidant vitamin in an amount of 2-25% w/w and 1-15% w/w of the composition respectively or in the ratio of 1:10 to 10:1.

In a preferred embodiment the ratio of the sulphur-containing amino acid or peptide and the antioxidant vitamin in the composition ranges from 0.2:1 to 1:0.1.

The pharmaceutical compositions of the present invention have a pH in the physiological range of less than 3.5, preferably less than 3.0.

In another aspect, the present invention provides a stable pharmaceutical composition of palbociclib, where the composition has high drug loading.

The term “high drug load” as used herein, refers from about 30% to about 90% by weight of palbociclib based on the total weight of the composition.

Unless otherwise recited or required by the context, percent and “%” refer to percent by weight.

The pharmaceutical compositions of present invention comprise about 10 mg to about 500 mg of palbociclib, preferably about 25 to about 200 mg of palbociclib. The pharmaceutical composition comprises palbociclib in the range of about 40% to about 90% by weight on the basis of the total weight of the composition.

In accordance with still another embodiment of the present invention, there is provided a high drug load pharmaceutical composition which is stable at 40° C. and 75% relative humidity.

As further described herein, the present invention provides solid dosage forms comprising palbociclib, at least one compound selected from the group consisting of sulphur-containing amino acid or peptide, at least one vitamin having antioxidant properties, and one or more pharmaceutically acceptable excipients.

Solid dosage forms include, but are not limited to, immediate release tablets and capsules, controlled-release (CR) tablets and capsules, fast-dissolve dosage forms, chewable dosage forms, sachets, etc. Preferably, the dosage form of the present invention is in the form of a tablet, including monolayer or bilayer tablets.

A “solid dosage form” of the present invention is a pharmaceutically-acceptable solid dosage form that is safe for oral administration to humans, where all excipients in the dosage form are pharmaceutically acceptable for use in oral formulations, in other words safe for human ingestion. In frequent embodiments, the solid dosage form is a tablet.

In frequent embodiments of each of the aspects described herein, the solid dosage form of the invention is in the form of a tablet. In some embodiments, the tablet is film coated. In some embodiments, the tablet is a monolayer tablet. In other embodiments, the tablet is a bilayer tablet.

The term “excipient” means a pharmacologically inactive component such as a diluent, lubricant, surfactant, carrier, or the like. The excipients that are useful in preparing a pharmaceutical composition are generally safe, non-toxic and are acceptable for veterinary as well, as human pharmaceutical use. Reference to an excipient includes both one and more than one such excipient. Co-processed excipients are also covered under the scope of present invention.

In another embodiment of the present invention there is provided a stable solid oral pharmaceutical composition comprising palbociclib with one or more pharmaceutically acceptable excipient and/or carrier like diluent, binder, disintegrant, surfactant, wetting agent, lubricant, pH adjusting agents, coloring agent, sweetening agents, flavoring agent, buffers, and other pharmaceutical excipients.

Various useful fillers or diluents include, but are not limited to calcium carbonate, calcium phosphate, dibasic anhydrous, calcium phosphate, dibasic dihydrate, calcium phosphate tribasic, calcium sulphate, cellulose powdered, silicified microcrystalline cellulose, cellulose acetate, compressible sugar, confectioner's sugar, dextrates, dextrose, fructose, lactitol, lactose, magnesium carbonate, magnesium oxide, maltodextrin, maltose, mannitol, microcrystalline cellulose, polydextrose, simethicone, sodium alginate, sodium chloride, sorbitol, starch, pregelatinized starch, sucrose, trehalose and xylitol, or mixtures thereof. Preferably, filler is used in an amount of from about 1% to about 90% by weight. More preferably, the amount of diluent(s) may vary within a range of from about 0% to less than about 75% by weight based on the total weight of the composition.

Various useful binders include, but are not limited to acacia, alginic acid, carbomer, carboxymethylcellulose sodium, ceratonia, dextrin, dextrose, gelatin, hydroxyethyl cellulose, hydroxyethylmethyl cellulose, hydroxypropyl cellulose, low substituted hydroxypropyl cellulose, hypromellose, magnesium aluminium silicate, maltodextrin, maltose, methylcellulose, microcrystalline cellulose, polydextrose, polyethylene oxide, povidone, sodium alginate, starch, pregelatinised starch, stearic acid, sucrose and zein, or mixtures thereof. The amount of binder(s) may vary within a range of from about 0% to about 10% by weight based on the total weight of the composition. Preferably, binder is optionally used in an amount of about 0% to less than about 5% by weight.

Various useful disintegrants include, but are not limited to, alginic acid, calcium phosphate, tribasic, carboxymethylcellulose calcium, carboxymethylcellulose sodium, croscarmellose sodium, crospovidone, docusate sodium, guar gum, low substituted hydroxypropyl cellulose, magnesium aluminum silicate, methylcellulose, microcrystalline cellulose, povidone, sodium alginate, sodium starch glycolate, polacrilin potassium, silicified microcrystalline cellulose, starch or pre-gelatinized starch, or mixtures thereof. The amount of disintegrant(s) may vary within a range of from about 0% to about 15% by weight based on the total weight of the composition. Preferably, disintegrant is optionally used in an amount of about 0% to less than about 5% by weight.

Lubricants used in the composition include, but are not limited to, calcium stearate, glycerine monostearate, glyceryl behenate, glyceryl palmitostearate, hydrogenated castor oil, hydrogenated vegetable oil 0 type I, magnesium lauryl sulphate, magnesium stearate, medium-chain triglycerides, poloxamer, polyethylene glycol, sodium benzoate, sodium chloride, sodium lauryl sulphate, sodium stearyl fumarate, stearic acid, talc, sucrose stearate and zinc stearate or mixtures thereof. Preferably, lubricant is present in an amount of about 0% to about 5% by weight. More preferably, lubricant is present in an amount of about 0.1% to less than about 2% by weight.

Glidants improve flowability and accuracy of dosing. However, known glidants like tribasic calcium phosphate and colloidal silicon dioxide are not present in the compositions prepared as per present invention.

Various directly compressible grades of pharmaceutically acceptable excipients are also contemplated within the scope of the present invention. Directly compressible excipients include but are not limited to anhydrous lactose, spray dried lactose, dibasic calcium phosphate dihydrate, microcrystalline cellulose, powdered cellulose, low substituted hydroxypropyl cellulose, dextrose, sucrose, spray dried maltose, maltodextrin, mannitol, xylitol, sorbitol, lactitol, starch, pregelatinized starch etc.

Surfactants or surface-active agents improve wettability of the dosage form and/or enhance its dissolution. Surfactants contemplated in the present invention include but are not limited to anionic surfactants, amphoteric surfactants, non-ionic surfactants and macromolecular surfactants. Suitable examples of surfactants include but are not limited to sodium lauryl sulphate, sodium cetyl stearyl sulphate or sodium dioctyl sulphosuccinate, lecithin, cetyl alcohol, stearyl alcohol, cetyl stearyl alcohol, cholesterol, sorbitan fatty acid esters such as sorbitan mono-oleate, polyoxyethylene sorbitan fatty acid esters such as polysorbate 20, polyoxyethylene fatty acid glycerides such as macrogol 1000 glycerol monostearate, polyoxyethylene fatty acid esters such as polyoxyl 40 stearate, polyoxyethylene fatty alcohol ethers such as polyoxyl 10 oleyl ether, glycerol fatty acid esters such as glycerol monostearate. Suitable example of a macromolecular surfactant includes but is not limited to Poloxamer. Preferably, the surfactant is used in an amount of about 0% to less than about 5% by weight.

Various film forming agents include but are not limited to cellulose derivatives such as soluble alkyl- or hydroalkylcellulose derivatives such as methylcellulose, hydroxymethyl cellulose, hydroxyethyl cellulose, hydroxypropyl cellulose, hydroxymethylethyl cellulose, hydroxypropyl methylcellulose, sodium carboxymethyl cellulose, etc., acidic cellulose derivatives such as cellulose acetate phthalate, cellulose acetate trimellitate, and methylhydroxypropylcellulose phthalate, polyvinyl acetate phthalate, etc., insoluble cellulose derivative such as ethylcellulose and the like, dextrins, starches and starch derivatives, polymers based on carbohydrates and derivatives thereof, gum arabic, xanthans, alginates, polyvinyl alcohol, polyvinyl acetate, polyvinylpyrrolidone, chitosan and derivatives thereof, shellac and derivatives thereof, waxes and fat substances.

If desired, the films may contain additional adjuvants for coating such as plasticizers, polishing agents, colorants, pigments, antifoaming agents, opacifiers, and the like. As an alternative to the above coating ingredients, pre-formulated coating products such as Opadry™ may be used. The products that are sold in dry form require only mixing with a liquid before use.

In another preferred embodiment, the pharmaceutical composition of the present invention comprises palbociclib 15-25% w/w, sulphur-containing amino acid 5-25% w/w, antioxidant vitamin 5-25% w/w, filler 40-60% w/w, disintegrant 2-8% w/w and lubricant 4-7% w/w.

In another aspect, the invention provides a method of treating cancer comprising administering to a subject in need thereof a therapeutically effective amount of the solid dosage form of any of the aspects and embodiments described herein. In particular embodiments, the cancer is breast cancer. Palbociclib may be administered alone or in combination with other drugs, in particular aromatase inhibitors, e.g., letrozole, fulvestrant or exemestane, and will generally be administered as a formulation in association with one or more pharmaceutically acceptable excipients.

In yet another aspect the present invention provides compositions comprising palbociclib, wherein the composition has comparable in-vitro dissolution profile similar to that of IBRANCE® tablets.

An embodiment of the present invention provides a solid dosage form of any of the embodiments described herein, wherein the dosage form when added to a test medium comprising 500 mL of 10 mM pH 5.5 acetate buffer at 37° C. in a standard USP 2 rotating paddle apparatus with the paddles spinning at 50 rpm dissolves: (a) not less than 35% of the palbociclib in 15 minutes; (b) not less than 45% of the palbociclib in 30 minutes; (c) not less than 55% in 60 minutes; or (d) two or more of (a), (b) and (c).

In another embodiment of the invention provides a solid dosage form of any of the embodiments described herein, wherein the dosage form when added to a test medium comprising 500 mL of 50 mM pH 6.5 phosphate buffer and 0.1 M NaCl at 37° C. in a standard USP 2 rotating paddle apparatus with the paddles spinning at 50 rpm dissolves: (a) not less than 15% of the palbociclib in 15 minutes; (b) not less than 20% k of the palbociclib in 30 minutes; (c) not less than 25% of the palbociclib in 60 minutes; or (d) two or more of (a), (b) and (c).

In yet another embodiment, the invention provides a solid dosage form, wherein the dosage form: (a) has a mean fed/fasted ratio of the area under the plasma concentration versus time curve (AUC) from about 0.8 to about 1.25 after administration of a single oral dose to a subject; (b) has a mean fed/fasted ratio of the maximum plasma concentration (Cmax) from about 0.8 to about 1.25 after administration of a single oral dose to a subject; or (c) both (a) and (b).

In yet another embodiment, the invention provides a solid dosage form, wherein the dosage form: (a) provides a mean fasted AUC in the range of 80% to 125% of the mean fasted AUC for a control immediate release (IR) oral capsule containing an equivalent amount of palbociclib after administration of a single oral dose to a subject; or (b) provides a mean fasted Cmax in the range of 80% to 125% of the mean fasted Cmax for a control immediate release (IR) oral capsule containing an equivalent amount of palbociclib after administration of a single oral dose to a subject; or (c) both (a) and (b).

In a fifth aspect the present invention provides palbociclib formulations for oral administration, by a manufacturing process which is consistent and therefore feasible for industrial production, while maintaining stability and pharmaceutical equivalence to the reference listed drug.

The process of the present invention besides being simple, reproducible, cost-effective and stable alternate dosage form of palbociclib which offers desirable technical attributes such as disintegration, dissolution, improved flow characteristics such as bulk density, tapped density, stability, bioequivalence comparable to the commercially available counterpart (IBRANCE® tablets).

In accordance with still another embodiment of the present invention, there is provided a stable pharmaceutical composition comprising palbociclib prepared by wet granulation, dry granulation, dry blending, dry mixing or direct compression process. In another embodiment of the invention, the pharmaceutical composition comprising palbociclib is prepared by wet or dry process. The wet and dry processes include, but are not limited to, wet granulation, dry granulation, dry blending, dry mixing and direct compression. Other formulation techniques are also contemplated within the scope of the present invention. Any pharmaceutically acceptable granulating agent can be used for wet granulation. Preferable granulating solvents include, but are not limited to, water, esters such as ethyl acetate; ketones such as acetone; alcohols such as methanol, ethanol, isopropanol, butanol; dichloromethane, chloroform, dimethyl acetamide (DMA), dimethyl sulfoxide (DMSO), ether, diethyl ether and combinations thereof.

In another embodiment of the invention, wet granulation can be performed using Rapid mixer granulator, Fluid bed granulator, Planetary mixer and the like; dry blending can be performed using V-blender or key blender; and dry granulation can be performed using roller compacter or slugging techniques or by any other method known in the art.

In accordance with still another embodiment of the present invention, there is provided a process for the preparation of a stable pharmaceutical composition comprising palbociclib or its pharmaceutically acceptable salts, esters, solvates, polymorphs, enantiomers or mixtures thereof comprising the steps of:

a) Sifting the accurately weighed quantities of active agent and one or more pharmaceutically acceptable excipient(s) through a suitable sieve followed by mixing;

b) Granulating the mixture of step a) with a binder solution (aqueous or non-aqueous solvent)

c) Drying the granulated mass, optionally milling of the dried granules;

d) optionally mixing with other pharmaceutical acceptable excipients to prepare granule dosage form or optionally compressing the granules to form tablets.

In another embodiment of the invention, there is provided a process for preparation of immediate release dosage form of palbociclib wherein the process is easily scalable at an industrial scale.

The principles, preferred embodiments, and modes of operation of the present invention have been described in the foregoing specification. The invention which is intended to be protected herein, however, is not to be construed limited to the particular forms disclosed, since these are to be regarded as illustrative rather than restrictive. Variations and changes may be made by those skilled in the art, without departing from the spirit of the invention.

### EXAMPLES

### Example 1: Film-Coated Tablet Composition of Palbociclib

| Component | % w/w |
|---|---|
| Palbociclib | 40.0 |
| Microcrystalline cellulose | 30.0 |
| Colloidal silicon dioxide | 0.95 |
| L-cysteine | 9.78 |
| Ascorbic acid | 9.78 |
| Crospovidone | 5.5 |
| Magnesium stearate | 3.44 |
| Purified water | Q.s. |

Procedure:
1. Palbociclib and microcrystalline cellulose were co-sifted through #10 ASTM.
2. Copovidone and colloidal silicon dioxide were co-sifted through #20 ASTM.
3. Sifted materials obtained in step 1) and 2) were loaded into blender for mixing for 20 min.
4. Magnesium stearate was sifted through #60 ASTM and added to the blender in step 3) and mixed for 5 minutes followed by roller compaction and milling.
5. L-cysteine, ascorbic acid, microcrystalline cellulose and copovidone were weighed and passed through #30 ASTM mesh. Magnesium stearate was weighed and passed through #60 ASTM mesh.
6. Extragranular materials of step 5) were added to the milled granules obtained in step 4) and mixed, followed by compression and film coating.

### Example: 2 Film-Coated Tablet Composition of Palbociclib

| Component | % w/w |
|---|---|
| Palbociclib | 52.5 |
| Microcrystalline cellulose | 10.5 |
| Colloidal silicon dioxide | 0.95 |
| Glutathione | 10.25 |
| α-tocopherol | 15.75 |
| Crospovidone | 4.55 |
| Magnesium stearate | 5.5 |
| Purified water | Q.s. |

Procedure:
1. Palbociclib and microcrystalline cellulose were co-sifted through #10 ASTM.
2. Copovidone and colloidal silicon dioxide were co-sifted through #20 ASTM.
3. Sifted materials obtained in step 1) and 2) were loaded into blender for mixing for 20 min.
4. Magnesium stearate was sifted through #60 ASTM and added to the blender in step 3) and mixed for 5 minutes followed by roller compaction and milling.
5. Glutathione, α-tocopherol, microcrystalline cellulose and copovidone were weighed and passed through #30 ASTM mesh. Magnesium stearate was weighed and passed through #60 ASTM mesh.
6. Extragranular materials of step 5) were added to the milled granules obtained in step 4) and mixed, followed by compression and film coating.

### Example: 3 Dissolution Studies

Test tablets prepared as described in Example-1 were subjected to dissolution testing using USP Apparatus II with paddles spinning at 50 RPM in 900 ml of phosphate buffer at pH 6.8 as dissolution media and the results are tabulated below:

TABLE 1

| Time | Percentage (%) of Drug |
|---|---|
| (in min) | Released |
| 0 | 0 |
| 5 | 13 |
| 10 | 31 |
| 15 | 39 |
| 20 | 51 |
| 30 | 53 |
| 45 | 54 |
| 60 | 55 |

The tablets exhibited desirable dissolution performance, with greater than 50% of the drug dissolved at 30 minutes as shown in the Table-1 above.

### Example: 3 Chemical Stability of Formulations

The test tablets prepared in Example-1 were stored at 70° C./75% RH for 8 days. The tablets were crushed and analyzed for impurities using a high-performance liquid chromatography (HLPC) method. The tablets were found to have acceptable total impurities after storage at 70° C./75% RH for 8 days.

The principles, preferred embodiments, and modes of operation of the present invention have been described in the foregoing specification. The invention which is intended to be protected herein, however, is not to be construed limited to the particular forms disclosed, since these are to be regarded as illustrative rather than restrictive. Variations and changes may be made by those skilled in the art, without departing from the spirit of the invention. cm We claim:

1. A solid oral dosage form comprising:
i. palbociclib,
ii. at least one compound selected from the group consisting of sulphur-containing amino acid or peptide,
iii. at least one vitamin having antioxidant properties, and
iv. one or more pharmaceutically acceptable excipients.

2. The solid oral dosage form as claimed in claim 1, wherein the sulphur-containing amino acid or peptide is selected from the group consisting of cysteine, methionine, aminoethylsulfonic acid (taurine), glutathione, cystine, homocysteine, homocystin, cysteine sulfinic acid, lanthionine, mixtures thereof.

3. The solid oral dosage form as claimed in claim 1, wherein the sulphur-containing amino acid or peptide is L-cysteine.

4. The solid oral dosage form as claimed in claim 1, wherein the vitamin having antioxidant properties is selected from the group consisting of vitamin C, vitamin E, vitamin A, vitamin-like active substances, mixtures thereof.

5. The solid oral dosage form as claimed in claim 1, wherein the sulphur-containing amino acid or peptide is selected from the group consisting of ascorbic acid, metal ascorbate, for example sodium ascorbate, potassium ascorbate, calcium ascorbate, magnesium ascorbate, aluminium ascorbate, ascorbic acid derivatives.

6. The solid oral dosage form as claimed in claim 1, wherein the amount of sulphur-containing amino acid or peptide to the antioxidant vitamin are in a ratio of 1:10 to 10:1.

7. The solid oral dosage form as claimed in claim 1, wherein palbociclib comprises about 40% to about 90% w/w of the composition.

8. The solid oral dosage form as claimed in claim 1, wherein the composition comprises one or more pharmaceutically acceptable excipients selected from the group consisting of carrier, diluent, binder, disintegrant, surfactant, wetting agent, lubricant, pH adjusting agents, coloring agent, sweetening agents, flavoring agent, buffers, and mixtures there of.

9. The solid oral dosage form as claimed in claim 1, wherein the dosage form is a tablet.
